(19) United States
(12) Patent Application Publication
(10) Pub. No.: US 2005/0154081 A1
(43) Pub. Date: Jul. 14, 2005

(54) OPACITY AND COLOR CHANGE POLYMERIZABLE DENTAL MATERIALS

(75) Inventors: Rui YIN, Buffalo Grove, IL (US); Byoung I. SUH, Oak Brook, IL (US); Oanh MAI, North Aurora, IL (US); Keith A. HAYES, Lombard, IL (US); Louis J. SHARP, Libertyville, IL (US)

Correspondence Address: HOWREY SIMON ARNOLD & WHITE LLP, c/o IP DOCKETING DEPARTMENT, 2941 FAIRVIEW PARK DRIVE, SUITE 200, FALLS CHURCH, VA, 22042-7195, US

(73) Assignee: BISCO, INC., SchaumbUrg, IL (US)

(21) Appl. No.: 10/707,762

(22) Filed: Jan. 9, 2004

(51) Int. Cl. A61F 2/00

(52) U.S. Cl. 523/115

(57) ABSTRACT

Polymerizable compositions are described containing colored polymerization indicators which confer a visible color on the unpolymerized compositions. Upon polymerization, the compositions exhibit reduced or eliminated color. The compositions can also change their opacity during polymerization. The compositions provide easy visual differentiation between the unpolymerized compositions and substrates on which the compositions are placed. The compositions are useful in clinical and laboratory applications such as those involving teeth, dental implants, orthodontic materials, artificial teeth and bones.

### BACKGROUND OF INVENTION

1. Filed of the Invention

The invention relates to polymerizable dental materials, and more specifically, to polymerizable dental and bone materials that change color and/or translucency when cured.

2. Description of Related Art

Polymerizable dental compositions are widely used in clinical settings. Polymerizable compositions having a polymerizable monomer component and a relatively high amount of inorganic filler component of suitably sized particles can be used in fillings, pit and fissure sealants, and in dental restorations such as bridges, crowns, and dentures where resistance to compressive and abrasive forces are desireable. Polymerizable compositions with a relatively lower filler content of suitable sized particles and a polymerizable monomer component can be used a dental adhesive compositions useful in securing composite materials or other dental appliances to each other and/or to tooth structure. The aforementioned dental compositions can contain monomers which are polymerized by the dentist or technician (e.g. by light, self-cure, or dual-cure). Dental composite materials typically contain one or more polymerizable monomers, a polymerization initiator, and optionally, fillers, colorants, fluoride release agents, or other additives.

For aesthetic and cosmetic reasons, dental patients prefer that the materials have a color and appearance identical to or similar to that of natural teeth. This similarity in appearance presents a challenge to the dentist, as it is sometimes difficult to visually distinguish the applied composite materials from the surrounding tooth material.

Various U.S. Patents have focused on preparing composite materials with improved color stability, minimizing color change over time after application and curing of the materials.

U.S. Pat. No. 3,991,008 (issued Nov. 9, 1976) suggests the use of a redox catalyst system consisting of a hydroperoxide oxidizing agent and a substituted thiourea reducing agent. The prepared materials did not exhibit color changes after prolonged exposure to ultraviolet light.

U.S. Pat. No. 5,684,103 (issued Nov. 4, 1997) suggests the use of bisazo initiators instead of peroxide initiators in order to avoid discoloration in aged dental restorative materials.

U.S. Pat. No. 6,281,265 (issued Aug. 28, 2001) offered that the addition of water-insoluble anti-microbial agents to curable dental compositions did not significantly affect the color of the compositions, and actually improved the long-term color stability of the cured compositions.

Several formulations have been discussed in the art to effect a change in color of dental materials after curing. These formulations typically involve the addition of a chemical compound specifically to effect the color change, and which does not otherwise participate in the curing process.

For example, U.S. Pat. No. 4,678,436 (issued Jul. 7, 1987) proposes a color changing cement composition for dental use. The composition contains a component that changes color with a change in pH caused by setting of the cement.

U.S. Pat. No.5,094,619 (issued Mar. 10, 1992) offers the addition of “color additives” (materials that gain color after application of an external stimulus) or “color subtractives” (materials that lose color after application of an external stimulus) to dental compositions. External stimuli listed include heat/cold, laser light, visible light, ultraviolet light, microwave radiation, and x-ray radiation. The external stimulus is applied after polymerization of the dental compositions to change the color of the compositions.

U.S. Pat. No.6,444,725 (issued Sep. 3, 2002) offers dental sealants comprising a hardenable (polymerizable) resin, a hardener (initiator), and a colorant (dye). The composition changes from an initial color to a final color after exposure to actinic radiation. The color change can occur subsequent to, or simultaneously with polymerization of the resin. A list of photo-bleachable colorant dyes is disclosed. The patent does not describe a change in opacity of the polymerized material.

Materials H-Nu 470, H-Nu 535, and H-Nu 635 are colored photoinitiators commercially available from Spectra Group Limited, Inc. (Maumee, Ohio). Various other colored fluorescers and photoinitiators are described in U.S. Pat. No. 5,395,862 (issued Mar. 7, 1995) and U.S. Pat. No. 5,451,343 (issued Sep. 19, 1995). The H-Nu products are described as photoinitiators that bleach during polymerization to provide a nearly clear cured product.

It is of continued value to have dental materials that have a non-tooth color prior to curing, and that subsequently have a different color after curing. It would be further desirable if the color after curing was similar or identical to that of natural teeth for cosmetic and aesthetic reasons.

### SUMMARY OF INVENTION

Polymerizable compositions are disclosed comprising a colored polymerization indicator which changes color during polymerization of the composition. The compositions can also or alternatively change opacity/translucency during polymerization. The compositions can include acrylate, methacrylate, and other polymerizable materials. The compositions can also include solvents, fillers, nanofillers, diluents, or other materials useful in dental formulations.

The compositions have an attractive feature of being colored and/or opaque (to the eye) when in an unpolymerized state, and have a reduced color or are uncolored and/or have a reduced opacity or are translucent after polymerization. The color change and/or opacity change compositions are useful to clinical and dental laboratory practitioners, as the compositions can easily be distinguished from tooth material during preparation and placement, but resemble the surrounding tooth material after polymerization. The change in color and/or opacity also serves as a “real time” visual indicator that the polymerization reaction is proceeding.

### BRIEF DESCRIPTION OF DRAWINGS

The following figures form part of the present specification and are included to further demonstrate certain aspects of the present invention. The invention may be better understood by reference to one or more of these figures in combination with the detailed description of specific embodiments presented herein.

FIG. 1 shows the reduction of color intensity of a composition containing a colored polymerization indicator (composition J; Example 13). A higher ΔE value represents a greater change in color.

FIG. 2 shows the reduction in opacity of a composition containing a colored polymerization indicator (composition B; Examples 5 and 18).

### DETAILED DESCRIPTION

Embodiments of the instant invention generally include un-polymerized compositions, polymerized compositions, methods of preparing the compositions, and methods of using the compositions in dental, bone, and other clinical and laboratory applications.

While compositions and methods are described in terms of “comprising” various components or steps, the compositions and methods can also “consist essentially of” or “consist of” the various components and steps.

Polymerizable Compositions

One embodiment of invention relates to polymerizable dental compositions. Polymerizable dental or bone compositions generally comprise at least a resin component, a filler component, a colored polymerization indicator, and a polymerization initiator.

The resin component can include one or more acrylate, diacrylate, multiacrylate (three or more acrylate functionalities per molecule), methacrylate, dimethacrylate, and multimethacrylate (three or more methacrylate functionalities per molecule) compounds. The resin component can comprise one or more acrylates, one or more methacrylates, a mixture of acrylates and methacrylates, vinyl monomers, or epoxy (epoxide) monomers. It is presently preferred that the resin component is relatively non-volatile. The resin component can generally be present at any suitable non-zero concentration. It is presently preferred that the resin component is present at a concentration of about 10 weight percent to about 90 weight percent of the composition. High concentrations of the resin component can be used for materials such as composites. Low concentrations of the resin component can be used for materials such as cements and liners. Specific examples of concentrations include about 10 weight percent, about 20 weight percent, about 30 weight percent, about 40 weight percent, about 50 weight percent, about 60 weight percent, about 70 weight percent, about 80 weight percent, about 90 weight percent, and ranges between any two of these values.

A suitable acrylate is caprolactone acrylate. Suitable diacrylates include tripropylene glycol diacrylate, ethoxylated bisphenol A diacrylate, polyethylene glycol diacrylate, epoxy diacrylate, and urethane diacrylate. A suitable triacrylate is trimethylolpropane triacrylate. A suitable tetraacrylate is ditrimethylolpropane tetraacrylate. Ethoxylated forms of such acrylates may be used, as they have relatively higher surface tension.

Suitable mono-methacrylates include methmethacrylate (MMA), methacryloyl ethylphosphate (MEP), tetrahydrolfurfuryl methacrylate (THFMA), hydroxyethyl methacrylate (HEMA), hydroxypropyl methacrylate (HPMA), methacrylic acid (MAA), phenoxyethyl methacrylate, isobornyl methacrylate, ethoxylated nonylphenol methacrylate, lauryl methacrylate, and tridecyl methacrylate.

Suitable dimethacrylates include triethyleneglycol dimethacrylate, bisphenol “A” diglycidyl methacrylate, ethoxylated bisphenol “A” dimethacrylate and urethane dimethacrylate.

Suitable multifunctional monomers may include 2,2-bis[4-(3 -methacryloxy-2-hydroxypropoxy)phenyl]-propane (BIS-GMA), Bisphenol A dimethacrylate (Bis “A” Dima), ethoxylated Bis “A” Dima (Dima), neopentylglycol dimethacrylate, decanediol-1,10-dimethacrylate, dodecanediol-1,12-dimethacrylate, 1,4-butanediol dimethacrylate, ethyleneglycol dimethacrylate, diethyleneglycol dimethacrylate, triethylene glycol dimethacrylate (TEGDMA), tetraethyleneglycol dimethacrylate), polyethyleneglycol dimethacrylate, propyleneglycol dimethacrylate, dipropyleneglycol dimethacrylate, tripropyleneglycol dimethacrylate, tetrapropyleneglycol dimethacrylate, polypropyleneglycol dimethacrylate, hexamethyleneglycol dimethacrylate, 2,2-bis(4-methacryloxyphenyl)propane, 2,2-bis(4-methacryloxyethoxyphenyl)propane, 1,2-bis(3-methacryloxy-2-hydroxypropoxy)ethane, CPDM-the reaction product of cyclopentane tetracarboxylic acid dianhydride and 2 moles of hydroxyethyl methacrylate (HEMA), tetrahydrofurfuryl cyclohexene dimethacrylate (TCDM)-the reaction product of Epiclon B-4400 (Dainippon Inc. and Chemicals Inc., Ft. Lee, N.J.) with 2 moles of HEMA, 2,2-bis(4- methacryloxyphenyl)-propane, 2-hydroxy- 1,3-dimethacryloxypropane, di-2-methacryloxyethyl-2,2,4-trimethylhexamethylene dicarbamate (UDMA), di-2-methacryloxyethylisophorone dicarbamate, and di-2-methacryloxyethyl-2,4- or 2,6-tolylene dicarbamate, Bis(2-methacryloyloxyethyl) phosphate (BisMEP), trimethylolpropane trimethacrylate (TMPTMA), tetramethacryloyloxyethylpyrophosphoric acid, Bis-GMA phosphate, and hexa-functional methacrylate ester.

The resin component can generally be present in the composition at any suitable non-zero concentration. For example, the resin component can be present at a concentration of about 10 weight percent to about 90 weight percent of the composition. Examples of resin concentration includes about 10 weight percent, about 20 weight percent, about 30 weight percent, about 40 weight percent, about 50 weight percent, about 60 weight percent, about 70 weight percent, about 80 weight percent, about 90 weight percent, and ranges between any two of these values. If the resin component comprises two or more compounds, then the combined weight percent of the two or more compounds can be determined. The hydrophilicity or hydrophobicity of the compositions can be adjusted by selection of appropriate hydrophilic or hydrophobic monomers in the resin component. For example, addition of HEMA will make the composition more hydrophillic, while addition of Bis-GMA to a HEMA-containing composition will make the composition more hydrophobic.

The compositions can comprise one or more fillers, nanofillers, fibers, glass particles, fluoride release agents, and/or other dental materials. Examples of such fillers include sintered glass, ground glass, Ox-50 (a fumed silicon dioxide filler commercially available from Degussa; Dusseldorf, Germany), silane-treated Ox-50, fiberglass fibers, and glass ionomer powder IXG 1944 RGW from Ferro Corporation (Cleveland, Ohio), which is also a fluoride release agent. The fillers can have a low refractive index in order to obtain high clarity of the unpolymerized and polymerized compositions.

The refractive index (RI) of a material is the change in direction of light rays passing from one medium to another of different density, as from air to water or glass. It is often described as the bending of light. When light travels through a medium and strikes another material of lower refractive index, the light is bent towards the material of higher refractive index. To produce a translucent material containing two or more components, the components are selected to have similar refractive indeces, even though each individually may appear translucent.

In general, the refractive index is a function of the molecular structure, functionality and molecular weight. A monomer with ring structure tends to have a higher refractive index than straight chain monomer. Monomers with high functionality tend to have high refractive indeces. However, structure plays a greater role in determining the refractive index than does the functionality. The refractive index increases as the molecular weight of a particular type of monomer increases (i.e. monomers with similar backbone structures, but varying in molecular weights).

Refractive index also varies with the wavelength of the incident light, temperature, and pressure. For example, the refractive index of Bisco Aeliteflo resin changes with the wavelength at 20° C. as shown in the following table.

TABLE 1
Refractive index by wavelength

| Wavelength (nm) | Refractive index |
|---|---|
| 404.6564 | 1.5673 |
| 435.8343 | 1.5600 |
| 479.9914 | 1.5527 |
| 546.0740 | 1.5454 |
| 587.5618 | 1.5422 |
| 643.8469 | 1.5390 |
| 706.5188 | 1.5362 |
| 852.1100 | 1.5321 |

Except for silicon fillers, most glass particles have higher refractive indeces than methacrylate monomer and even polymers. For examples, the refractive index of barium glass and strontium glass from Ferro Corporation (Cleveland, Ohio) are 1.556. In contrast, the refractive index of triethyleneglycol methacrylate (TEGDMA) is 1.4606.

TABLE 2
Refractive index of fillers

| Material | Refractive index |
|---|---|
| Aluminum oxide 500 | 1.76 |
| Sr glass (IX 2405 RWG; Ferro Corp.) | 1.556 |
| Sr glass (IX 2405 SRRG; Ferro Corp.) | 1.557 |
| Sr glass (Schott Corp.) | 1.523 |

TABLE 3
Refractive index of monomers and resin

| Material | Refractive index |
|---|---|
| Bis-GMA | 1.5500 |
| UDMA | 1.4843 |
| Bis-EMA (4-6 mole) | 1.5392 |
| TEGDMA | 1.4606 |
| THFMA | 1.4670 |
| HEMA | 1.4522 |
| TCDM | 1.4895 |
| BisMEP | 1.4688 |

The compositions can comprise filler(s) at generally any non-zero concentration suitable for use in dental and/or bone applications. For example, the total filler concentration can be about 90 weight percent to about 10 weight percent. Example total filler concentrations can include about 10 weight percent, about 20 weight percent, about 30 weight percent, about 40 weight percent, about 50 weight percent, about 60 weight percent, about 70 weight percent, about 80 weight percent, about 90 weight percent, and ranges between any two of these values.

The compositions also comprise at least one colored polymerization indicator. The colored polymerization indicator is colored (i.e. colored to the eye, absorbing light in the visible spectrum), and imparts a non-tooth color to the composition prior to polymerization. During polymerization of the composition, the colored polymerization indicator and the composition experience a reduction in the intensity of the color. Preferably, the polymerization results in a loss of the color imparted by the colored polymerization indicator. The reduction in intensity of the color can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and ideally about 100%. In other words, the color intensity of the polymerized composition can be less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the color intensity of the unpolymerized composition. Ideally, the color intensity of the unpolymerized composition is eliminated in the polymerized composition (i.e. the polymerized composition is uncolored, or is the color of teeth or bone).

Color can be quantitatively determined using conventional instruments such as the Hunter Ultrascan XE (Hunter Associates Laboratory; Reston, Va.). Colors can be represented by L, a, and b values. Generally any color that is easily visually distinguished from teeth or bone can be used. Example colors include red, pink, orange, yellow, green, blue, and purple.

Color change can be quantitatively expressed by ΔE. The ΔE value can be calculated to evaluate the total color change of a material using the following equation:
ΔE=Square root((L₁−L₀)²+(a₁−a₀)²+(b₁−b₀)²)

In the formula, the 0 subscript represents the state before curing, and the 1 subscript represents the state after curing. The reduction in color change as determined by ΔE can be at least about 10, at least about 20, at least about 30, at least about 40, at least about 50, at least about 60, at least about 70, at least about 80, at least about 90, and ranges between any two of these values.

The final color of the polymerized materials can be semi-quantitatively determined by comparing the color against a series of shade standards. One commonly used set of standards in the dental field is the VITA-shade standards (VITA is a registered trademark of Vita Zahnfabrik; Bad Sackingen, Germany). The shade system assigns letters (A, B, C, or D) and numbers (1-5 for A, 1-4 for B-D) to represent the color and darkness of a material, respectively.

Examples of such a colored polymerization indicator are H-NU470 (5,7-diiodo-3-butoxy-6-fluorone; 6-Butoxy-2,4-diiodo-xanthen-3-one; lambda max of 470 nm (orange color)), H-NU535 (6-Hydroxy-2,4,5,7-tetraiodo-xanthen-3-one; lambda max of 535 nm (red color)), and H-NU635 (6-Hydroxy-2,4,5,7-tetraiodo-3-oxo-3H-xanthene-9-carbonitrile; lambda max of 635 nm (blue color)), all commercially available from Spectra Group Limited, Inc. (Maumee, Ohio).

The colored polymerization indicator can generally be present at any suitable non-zero concentration. For example, the indicator can be present at a non-zero concentration of up to about 1 weight percent of the composition. Specific examples of indicator concentrations include about 0.001 weight percent, about 0.005 weight percent, about 0.01 weight percent, about 0.02 weight percent, about 0.03 weight percent, about 0.04 weight percent, about 0.05 weight percent, about 0.10 weight percent, about 0.15 weight percent, about 0.20 weight percent, about 0.25 weight percent, about 0.30 weight percent, about 0.35 weight percent, about 0.40 weight percent, about 0.45 weight percent, about 0.50 weight percent, about 0.55 weight percent, about 0.60 weight percent, about 0.65 weight percent, about 0.70 weight percent, about 0.75 weight percent, about 0.80 weight percent, about 0.85 weight percent, about 0.90 weight percent, about 0.95 weight percent, about 1 weight percent, and ranges between any two of these values.

The compositions can further comprise one or more polymerization initiators or co-initiators. The initiators can be uncolored or colored. The initiator can retain its color during the polymerization of the composition. Combinations of colored and uncolored initiators can be used. If colored initiators experience reduced performance during polymerization, a combination of colored and uncolored initiator(s) can be used to compensate for this effect. The uncolored initiator can be present at a non-zero concentration of up to about 2 weight percent of the composition. If multiple initiators are used, the total combined initiator concentration can be a non-zero concentration of up to about 2.5 weight percent of the composition. An initiator used in dental composites is camphorquinone (CQ). It has a yellow color that is partially, but not completely, photo-bleachable.

In one embodiment of the invention, the polymerized compositions can have a lower color intensity than the unpolymerized compositions, as described above. In an alternative embodiment, the polymerized compositions can have a lower opacity than the unpolymerized compositions (i.e. the polymerized compositions have a higher translucency/transparency than the unpolymerized compositions). In a presently preferred embodiment, the polymerized compositions have both a lower color intensity and a lower opacity than the unpolymerized compositions.

Opacity (or inversely, translucency) of the unpolymerized and polymerized compositions can be measured using a HunterLab UltraScan XE instrument. Opacity is commonly represented as a percentage value. The opacity of the unpolymerized and polymerized compositions can be compared to calculate the reduction in opacity. The reduction can be determined as an absolute “difference” value (i.e. initial opacity minus final opacity) or as a relative percent reduction (i.e. 100%×(initial opacity−final opacity)/initial opacity).

In one embodiment of the invention, the filler component has a different refractive index from the remaining fraction of the unpolymerized composition. This difference in refractive index values confers a partial or total opaque appearance on the unpolymerized composition. After polymerization, the difference in refractive index between the filler component and the remaining fraction of the polymerized composition is smaller than the difference observed in the unpolymerized composition. This reduction in the difference indicates that the polymerized composition has a reduction in opacity (i.e. appears more translucent or transparent to the eye).

The reduction in opacity can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and ideally about 100%. In other words, the opacity of the polymerized composition can be less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the opacity of the unpolymerized composition. The opacity of the unpolymerized composition can be eliminated in the polymerized composition (i.e. the polymerized composition is transparent). The final opacity value of the polymerized composition is preferably less than about 70% (actual value, not relative), but varies depending on the selected application. For example, posterior applications may have opacities of greater than 70% (actual), anterior can have 50-70% (actual), incisal can have 40-50% (actual), and clear pastes can have 40% (actual) or less.

The compositions can further comprise one or more fixed dyes or pigments. For example, aluminum lake (C₁₆H₉N₄O₉S₂Al) can be used to impart a yellow color; iron oxide red (Fe₂O₃ and Fe₃O₄) can be used to impart a red color; and iron oxide black (Fe₃O₄) can be used to impart a black color. Mixtures of fixed dyes or pigments can be used to further vary the color of the compositions.

The composition can further include a non-reactive solvent. Examples of such solvents include water, acetone, ethanol, and mixtures thereof.

The compositions can be radio-opaque in order to facilitate visualization of the compositions once placed in a dental or laboratory procedure. The compositions can be radio-opaque in the unpolymerized state, in the polymerized state, or in both the unpolymerized state and in the polymerized state. The compositions can be made radio-opaque by addition of at least one radio-opaque component. If desired, radio-opacity can be at least 1 mm aluminum according to the ISO 4049 standard. Radio-opacity can be conferred by selection of appropriate fillers (e.g. use of barium glass or strontium glass), or by the addition of additional materials such as barium zirconate, barium sulfate, or zirconium oxide.

The compositions preferably polymerize (“cure”) in a short period of time. Curing can be performed by any means sufficient to polymerize the compositions in an amount of time reasonable for clinical or laboratory procedures. Curing can include light polymerization, heat polymerization, redox, and self-polymerization. One presently preferred type of curing is light curing. The light curing can be performed at low light intensity or at high light intensity. Example light curing sources include Nd-YAG lasers, He—Ne lasers, diode lasers, light emitting diodes (LEDs), xenon lamps, and quartz tungsten halogen lamps.

The intensity of light is preferably an intensity suitable for use in a dental laboratory or in a dentist's office. Examples of light intensity ranges include less than about 50 mW/cm² less than about 100 mW/cm², about 200 mW/cm² or less, about 300 mW/cm² or less, about 400 mW/cm² or less, about 500 mW/cm² or less, about 600 mW/cm² or less, about 800 mW/cm² or less, and about 2000 mW/cm² or less, it being understood that higher light intensities can also be employed. Specific examples of light intensities include about 50 mW/cm², about 100 mW/cm², about 150 mW/cm², about 200 mW/cm², about 250 mW/cm², about 300 mW/cm², about 350 mW/cm², about 400 mW/cm², about 450 mW/cm², about 500 mW/cm², about 600 mW/cm², about 800 mW/cm², and about 2000 mW/cm². Higher light intensities may also be used. For example, Bisco's VIP™ Dental Light Curing system using a blue wavelength light source may be employed by the dentist. Light-curing systems for dental laboratories such as the Jeneric-Pentron Cure-Lite Plus light box system or the Triad light box system from Dentsply, Inc. may also be used for dental appliances. Bisco's Tescera™ ATL™ or Bisco's Tescera™ NTL™ System utilizing its light source without the nitrogen environment may also be used.

The time of light curing can generally be any time. Presently preferred time ranges include about two minutes or less, about one minute or less, less than about 30 seconds, less than about 20 seconds, less than about 15 seconds, less than about 10 seconds, and less than about 5 seconds. Specific examples of light curing times include about one minute, about 30 seconds, about 20 seconds, about 15 seconds, about 10 seconds, about 5 seconds, about 3 seconds, about 2 seconds, and about 1 second. Shorter light cure times are generally preferably to shorten patient time for the procedure and for the convenience of the dental practitioner.

Polymerized Compositions

An additional aspect of the invention is directed towards the material produced by polymerizing any of the above described compositions.

As described above regarding the unpolymerized compositions, the compositions can be described in part by their color change properties, opacity/translucency change properties, or by both their color change and opacity/translucency change properties. In one embodiment of the invention, the polymerized compositions can have a lower color intensity than the unpolymerized compositions, as described above. In an alternative embodiment, the polymerized compositions can have a lower opacity than the unpolymerized compositions (i.e. the polymerized compositions have a higher translucency/transparency than the unpolymerized compositions). In a presently preferred embodiment, the polymerized compositions have both a lower color intensity and a lower opacity than the unpolymerized compositions.

The polymerized compositions can exhibit a reduced intensity of color relative to the corresponding unpolymerized compositions. If the composition contains fixed dyes or other color-stable materials, the reduction would be calculated subtracting the color imparted by these color-stable materials.

Preferred polymerized composite compositions according to the present invention preferably have a Barcol hardness value of at least about 70 on the top and 65 on the bottom. Preferred polymerized bracket cement compositions according to the present invention preferably have a shear bond strength value of at least about 12 MPa for uncut enamel and orthodontic brackets, and more preferably at least about 18 MPa.

Methods of Use

Further aspects of the invention involve methods of using the above described unpolymerized compositions and polymerized compositions. The above described compositions can be used in a variety of clinical and dental laboratory applications. For example, the compositions can be used in or on a substrate such as a dental surface, a dental composite, a tooth, a dental implant, or an artificial tooth. Additional uses include use as a cement, as a liner, as an orthodontic bracket cement, or in preparing a lingual retainer.

Methods can comprise selecting a substrate, applying one of the above described unpolymerized compositions to the substrate, and polymerizing the unpolymerized composition to produce a polymerized composition. The polymerizing step can comprise applying heat or light to the composition. The methods can further comprise shaping the unpolymerized composition prior to the polymerizing step. Alternatively, the polymerizing step can comprise a short partial-polymerizing step, followed by a complete polymerizing step. After the partial-polymerizing step, the partially polymerized composition can be further shaped, or otherwise treated. Other materials such as fillers and reinforcement articles can be added to the compositions either prior to polymerization or after the partial-polymerizing step. The fully polymerized composition can be further treated by various methods such as grinding or polishing. The polymerized composition can be shaped to resemble the surface of the surrounding teeth or bone.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Materials and Units

The following list includes the abbreviations of materials used in these Examples, a description of the type of material, and a commercial source for the materials.

TABLE 4
Materials and commercial sources

| Material | Type of material | Commercial source |
|---|---|---|
| H-NU635 | Colored polymerization | Spectra Group Limited, |
| (tetraiodocyanohydroxyfluorone) | indicator | Inc. (Maumee, OH) |
| H-NU535 | Colored polymerization | Spectra Group Limited, |
| (tetraiodohydroxyfluorone) | indicator | Inc. (Maumee, OH) |
| H-NU470 (5,7-diiodo-3-butoxy- | Colored polymerization | Spectra Group Limited, |
| 6-fluorone) | indicator | Inc. (Maumee, OH) |
| CQ (camphorquinone) | Photosensitizer | Aldrich Chemical |
| | | (Milwaukee, WI) |
| Darocure | Photoinitiator | Sartomer (Exton, PA) |
| TPO | Photoinitiator | Ciba (Tarrytown, NY) |
| BHT (butylated hydroxytoluene) | Antioxidant | Aldrich Chemical |
| | | (Milwaukee, WI) |
| EDMAB (ethyl (4- | Initiator | Esschem (Linwood, PA) |
| dimethylamino)benzoate) | | |
| Methylhydroquinone (MEHQ) | Polymerization inhibitor | Aldrich Chemical |
| | | (Milwaukee, WI) |
| Ethoxylated Bis “A” Dima | Monomer | Sartomer (Exton, PA) |
| Bis-GMA (Bisphenol-A- | Monomer | Cook Composite (IL) |
| glycidyldimethacrylate) | | |
| BisMEP (Bis[2- | Monomer | Aldrich Chemical |
| {methacryloyloxy}ethyl] | | (Milwaukee, WI) |
| phosphate) | | |
| Triethleneglycoldimethyl | Monomer | Sartomer (Exton, PA) |
| methacrylate (TriEDMA) | | |
| DSDM | Monomer | Bisco, Inc.(Schauburg, |
| | | IL) |
| Glycidyl methacrylate | Monomer | Sartomer (Exton, PA) |
| Hydroxyethyl methacrylate | Monomer | Sartomer (Exton, PA) |
| Hydroxypropyl methacrylate | Monomer | Sartomer (Exton, PA) |
| 2,6-t-butyl-4-methyl phenol | Light stabilizer | |
| PEG200DMA | Monomer | Sartomer (Exton, PA) |
| TCDM | Monomer | Bisco, Inc.(Schauburg, |
| | | IL) |
| UDMA dimethacrylate | Monomer | Esschem (Linwood, PA) |
| Lumilus blue LZ | Fluorescing agent | Honeywell International |
| | | (Morristown, NJ) |
| SYLOID 74 | Colloidal silica | Grace Davison (Curtis |
| | | Bay, MD) |
| OX-50 | Fumed silicon dioxide | Degussa (Dusseldorf, |
| | filler | Germany) |
| Strontium Fluoroaluminosilicate | Filler containing fluoride | Schott Corp. (Elmsford |
| G018-090) | | NY) |
| R972 (AEROSIL R972) | hydrophobic fumed silica | Degussa (Dusseldorf, |
| | | Germany) |
| Quartz 5 μm | Fused Quartz | Industrial Corp. (Lionvill, |
| | | PA) |
| Raysorb T-4000 (Sr glass) | Strontium glass | Esschem (Linwood, PA) |
| Sodium Fluoride | Fluoride release | Aldrich Chemical |
| | | (Milwaukee, WI) |
| Milled fiber glass (GL-0259) | Fiberglass | MO-SCI Corp (Rolla, |
| | | MO) |
| Aluminum lake C₁₆H₉N₄O₉S₂Al | Pigment | Sun Chemical (Fort Lee, |
| | | NJ) |
| Iron oxide red | Pigment | Whittacker Clark & |
| Fe₂O₃ and Fe₃O₄ | | Daniels, Inc. (South |
| | | Palifiled, NY) |
| Iron oxide black | Pigment | Whittacker Clark & |
| Fe₃O₄ | | Daniels, Inc. (South |
| | | Palifiled, NY) |

Unless otherwise indicated, the concentrations of the various components are listed as weight percentages based on the total weight of the composition.

### Example 2

### Methods of Preparing Color Change Compositions

Unless otherwise indicated, all components of the color change compositions are prepared in four steps. First, stabilizers, initiators, colored polymerization indicator, and any tint colorants are added to the monomer(s) at room temperature with magnetic stirring. Mixing is continued until the components dissolve. This solution is added to any other monomers at room temperature with magnetic stirring. Mixing is continued until a homogeneous resin mixture is obtained. This mixing step can take up to several hours. Any fillers (e.g. glass or silica) and sodium fluoride are mixed by agitating with mixing stones on a laboratory scale, or with ball milling on a production scale. The resin mixture and filler mixture are combined in ratios to obtain a composite resin of desired viscosity. Laboratory scale batches are prepared by shearing the components using a mortar and pestle until a uniform, homogenous putty is obtained. Production scale batches are prepared by combining the components using mechanical mixers until a uniform, homogenous putty is obtained.

### Example 3

### Analysis and Characterization of Color Change Compositions

The compositions described herein can be characterized by a variety of methods and procedures.

Barcol hardness values can be determined using a 934-1 barcol hardness impressor instrument (Barber Colman Company; Loves Park, Ill.). The composite is loaded into a 2 mm deep stainless steel mold. The filled mold is placed between two mylar cover slips and pressed flat. The composite is polymerized by irradiating the top side with a dental curing light set at 500 mW/cm² for 20 seconds. The mylar cover slips are removed after two minutes. The barcol hardness of the top and bottom of the sample is measured using the impressor instrument.

Enamel shear bond strength values for orthodontic bonding can be determined using an Instron instrument. Extracted molars are set in acrylic such that a flat enamel surface is exposed and parallel to the force vector being applied by the Instron. Set molars are stored in 0.1% aqueous thymol solution until testing. The enamel surface is prophied with a pumice slurry for 15 seconds, and then rinsed with water and dried. A 37% phosphoric acid semi-gel (Etch-37; Bisco, Inc.; Schaumburg, Ill.) is applied onto the enamel surface for 15 seconds. The etched surface is rinsed and dried. The surface is frosty in appearance. Once coat of One-Step (Bisco, Inc.) is brushed onto the etched surface. The surface is dried for 5 seconds. Curing is performed with a VIP instrument (Bisco, Inc.) set at 500 mW/cm² for 10 seconds using a normal light guide, or for 5 seconds using a Power Slot tapered light guide. A small amount of the unpolymerized composite composition to be tested is applied to the mesh on the back of a bracket base. Curing is performed with a VIP instrument set at 500 mW/cm² for 20 seconds on the occlusal edge and 10 seconds on the gingival edge using a normal light guide, or for 5 seconds on both the occlusal edge and gingival edge (10 seconds total) using a Power Slot tapered light guide. The bonded enamel is immediately placed into 37 ° C deionized water for 2 hours. The brackets are sheared from the enamel substrate using an Instron instrument fitted with a blade. Crosshead speed is set at 5 mm/minute. The load in kilograms is recorded at the point of failure and used to calculate the Shear Bond Strength (“SBS”) in MPa. An average of five bonded samples is determined.

Color absorbance values and opacity values can be determined using a HunterLab UltraScan XE instrument (Hunter Associates Laboratory; Reston, Va.). A composite disk is prepared by loading composite into a 1.1 mm thick composite disk mold. The loaded mold is pressed between two glass plates treated with an antireflective coating. The sample is placed up to the port of a HunterLab UltraScan XE instrument. L, b, a and opacity values of the unpolymerized material are recorded by the instrument. The composite are polymerized thoroughly with a dental curing light through the glass plates. L, b, a and opacity values are recorded again with the glass plates still in place. Opacity values are typically represented by a percentage, where higher values are more opaque (i.e. 100% opacity is completely opaque, and 0% opacity is completely transparent). Unless otherwise indicated in the Examples, opacity values are represented as their actual numerical percentages, and not relative to an initial value of the composition. The L, b, a and opacity values are compared before and after cure. Color and opacity change may also apparent upon visual inspection.

The L*a*b* color system is based on a 3-dimensional color space with the positive X-axis representing red, the negative X-axis representing green, the positive Y-axis representing yellow, the negative Y-axis representing blue, and the Z-axis going from zero (black) to 100 (white) with the origin at 50. “L” values are black/white, “a” values are red/green, and “b” values are blue/yellow.

The refractive index of a material can be determined using a Fisher Abbe-31 Refractometer (Fisher Scientific International, Inc.; Hampton, N.H.) calibrated by 1-bromonaphthalene according to the manufacturer's directions.

### Example 4

### Preparation of Color Change Composition A

The following Table provides the components of example composition A.

TABLE 5
Composition A

| Component | Concentration |
|---|---|
| Hydroxypropyl methacrylate | 10.8000% |
| UDMA | 8.1000% |
| PEG200DMA | 6.5340% |
| CQ | 0.01620% |
| Darocure | 0.3670% |
| H-NU470 | 0.0040% |
| EDMAB | 0.8100% |
| MEHQ | 0.0050% |
| Light Stabilizer | 0.1600% |
| Schott Glass (G018-090) | 56.7200% |
| OX-50 | 9.0000% |
| SYLOID 74 | 5.0000% |
| R972 | 2.3380% |
| Total = | 100% |

### Example 5

### Preparation of Color Change Composition B

The following Table provides the components of example composition B.

TABLE 6
Composition B

| Component | Concentration |
|---|---|
| Hydroxyethyl methacrylate | 9.0400% |
| Bis-GMA | 5.5000% |
| PEG200DMA | 7.7520% |
| CQ | 0.0228% |
| Darocure | 0.3420% |
| H-NU470 | 0.0015% |
| EDMAB | 0.1370% |
| BHT | 0.0067% |
| Light Stabilizer | 0.1600% |
| Schott Glass (G018-090) | 66.0300% |
| R972 | 10.9700% |
| 0.05% Blue | 0.0380% |
| Total = | 100% |

### Example 6

### Preparation of Color Change Composition C

The following Table provides the components of example composition C.

TABLE 7
Composition C

| Component | Concentration |
|---|---|
| Hydroxyethyl methacrylate | 9.6000% |
| Bis-GMA | 1.8600% |
| BisMEP | 7.2000% |
| PEG200DMA | 4.8000% |
| CQ | 0.0243% |
| Darocure | 0.3630% |
| H-NU470 | 0.0015% |
| EDMAB | 0.1440% |
| BHT | 0.0072% |
| Light Stabilizer | 0.1600% |
| Schott Glass (G018-090) | 65.4000% |
| R972 | 7.0000% |
| SYLOID 74 | 3.4400% |
| Total = | 100% |

### Example 7

### Preparation of Color Change Composition D

The following Table provides the components of example composition D.

TABLE 8
Composition D

| Component | Concentration |
|---|---|
| Hydroxyethyl methacrylate | 9.6000% |
| Bis-GMA | 1.8600% |
| TCDM | 7.2000% |
| PEG200DMA | 4.8000% |
| CQ | 0.0243% |
| Darocure | 0.3630% |
| H-NU470 | 0.0015% |
| EDMAB | 0.1440% |
| BHT | 0.0072% |
| Light Stabilizer | 0.1600% |
| Schott Glass (G018-090) | 65.4000% |
| R972 | 7.0000% |
| SYLOID 74 | 3.4400% |
| Total = | 100% |

### Example 8

### Preparation of Color Change Composition E

The following Table provides the components of example composition E.

TABLE 9
Composition E

| Component | Concentration |
|---|---|
| Hydroxyethyl methacrylate | 9.6000% |
| Bis-GMA | 1.8600% |
| DSDM | 7.2000% |
| PEG200DMA | 4.8000% |
| CQ | 0.0243% |
| Darocure | 0.3630% |
| H-NU470 | 0.0015% |
| EDMAB | 0.1440% |
| BHT | 0.0072% |
| Light Stabilizer | 0.1600% |
| Schott Glass (G018-090) | 65.4000% |
| R972 | 7.0000% |
| SYLOID 74 | 3.4400% |
| Total = | 100% |

### Example 9

### Preparation of Color Change Composition F

The following Table provides the components of example composition F.

TABLE 10
Composition F

| Component | Concentration |
|---|---|
| Glycidyl methacrylate | 10.7700% |
| Bis-GMA | 3.8300% |
| PEG200DMA | 10.2000% |
| CQ | 0.0243% |
| Darocure | 0.3630% |
| H-NU470 | 0.0015% |
| EDMAB | 0.1440% |
| BHT | 0.0072% |
| Light Stabilizer | 0.1600% |
| Schott Glass (G018-090) | 67.5000% |
| R972 | 1.0000% |
| OX-50 | 6.0000% |
| Total = | 100% |

### Example 10

### Preparation of Color Change Composition G

The following Table provides the components of example composition G.

TABLE 11
Composition G

| Component | Concentration |
|---|---|
| Hydroxyethyl methacrylate | 10.7400% |
| UDMA | 5.1000% |
| Glycidyl Methacrylate | 8.9700% |
| CQ | 0.0243% |
| Darocure | 0.3560% |
| H-NU470 | 0.0015% |
| EDMAB | 0.1410% |
| BHT | 0.0072% |
| Light Stabilizer | 0.1600% |
| Schott Glass (G018-090) | 67.5000% |
| R972 | 1.0000% |
| OX-50 | 6.0000% |
| Total = | 100% |

### Example 11

### Preparation of Color Change Composition H The Following Table Provides the Components of Example Composition H.

TABLE 12
Composition H

| Component | Concentration |
|---|---|
| Glycidyl methacrylate | 8.9300% |
| Bis-GMA | 5.1000% |
| Hydroxyethyl Methacrylate | 10.7700% |
| CQ | 0.0243% |
| Darocure | 0.3630% |
| H-NU470 | 0.0015% |
| EDMAB | 0.1440% |
| BHT | 0.0072% |
| Light Stabilizer | 0.1600% |
| Schott Glass (G018-090) | 67.5000% |
| R972 | 1.0000% |
| OX-50 | 6.0000% |
| Total = | 100% |

### Example 12

### Preparation of Color Change Composition I

The following Table provides the components of example composition I.

TABLE 13
Composition I

| Component | Concentration |
|---|---|
| Hydroxyethyl methacrylate | 10.8100% |
| Bis-GMA | 1.2700% |
| 10 mole Ethoxylate | 2.5200% |
| PEG200DMA | 10.2000% |
| CQ | 0.0243% |
| Darocure | 0.3630% |
| H-NU470 | 0.0015% |
| EDMAB | 0.1440% |
| BHT | 0.0072% |
| Light Stabilizer | 0.1600% |
| Schott Glass (G018-090) | 67.5000% |
| R972 | 1.0000% |
| OX-50 | 6.0000% |
| Total = | 100% |

### Example 13

### Preparation and Testing of Blue Colored Composition J

The following TriEDMA solution, resin, powder, and paste were prepared and mixed at room temperature.

TABLE 14
TriEDMA solution

| Component | Concentration |
|---|---|
| TriEDMA (BHT) | 93.67% |
| BHT | 0.03% |
| UV-3000 | 2.00% |
| EDMAB | 3.00% |
| CQ | 0.50% |
| TPO | 0.50% |
| H-NU635 | 0.30% |
| Total | 100.0% |

TABLE 15
Resin

| Component | Concentration |
|---|---|
| BisGMA | 20.00% |
| UDMA | 40.00% |
| TriEDMA solution | 40.00% |
| Total | 100.0% |

TABLE 16
Powder

| Component | Concentration |
|---|---|
| T-4000 (RC) | 52.25% |
| Quartz 5 μm (RC) | 17.00% |
| Fiberglass (RC) | 10.00% |
| OX-50 (RC) | 15.00% |
| R972 | 5.00% |
| NaF | 0.75% |
| Total | 100.0% |

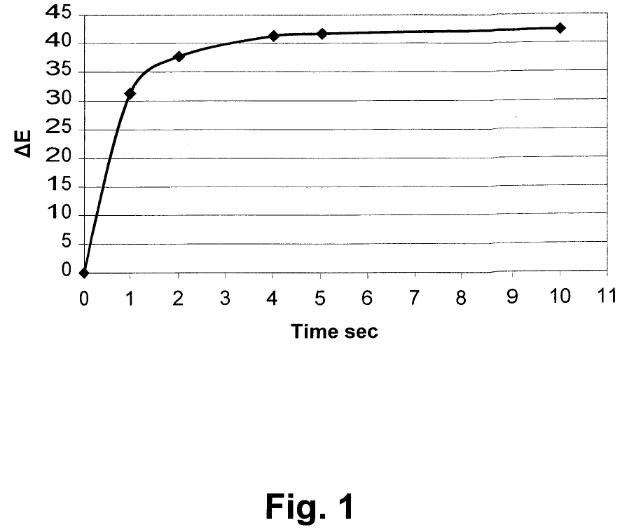

The paste blue composition J was prepared by mixing 83.00% powder and 17.00% resin at room temperature. Testing of samples showed an SBS Normal Light Guide value of 18.29±4.4 MPa, and an SBS Turbo Tip value of 13.59±5.1. The composition was polymerized, and the change in color is graphically represented in FIG. 1 (as shown by plotting the ΔE value against curing time). A ΔE value of greater than 40 was achieved after about 3 seconds of polymerization.

### Example 14

### Preparation and Testing of Composition K (“Lightbond™”) The following TriEDMA Solution, Resin, Powder, and Paste were Prepared and Mixed at Room Temperature

TABLE 17
TriEDMA solution

| Component | Concentration |
|---|---|
| TriEDMA (BHT) | 94.47% |
| BHT | 0.03% |
| UV-3000 | 2.00% |
| EDMAB | 3.00% |
| CQ | 0.50% |
| Total | 100.0% |

TABLE 18
Resin

| Component | Concentration |
|---|---|
| BisGMA | 20.00% |
| UDMA | 40.00% |
| TriEDMA solution | 40.00% |
| Total | 100.0% |

TABLE 19
Powder

| Component | Concentration |
|---|---|
| T-4000 (RC) | 62.25% |
| Quartz 5 μm (RC) | 17.00% |
| OX-50 (RC) | 15.00% |
| R972 | 5.00% |
| NaF | 0.75% |
| Total | 100.0% |

The paste composition K (Lightbond) was prepared by mixing 84.00% powder and 16.00 % resin at room temperature. Testing of samples showed an Ortho SBS with Power slot 5s/5s value of 17.93±5.8 MPa, and an Ortho SBS with Normal Light Guide value of 19.83±2.0 MPa.

### Example 15

### Preparation and Testing of Composition L (“Quickcure™”)

The following TriEDMA solution, resin, powder, and paste were prepared and mixed at room temperature.

TABLE 20
TriEDMA solution

| Component | Concentration |
|---|---|
| TriEDMA (BHT) | 87.28% |
| BHT | 0.03% |
| UV-3000 | 2.00% |
| EDMAB | 5.90% |
| CQ | 1.39% |
| Darocure 4265 | 3.4% |
| Total | 100.0% |

TABLE 21
Resin

| Component | Concentration |
|---|---|
| BisGMA | 20.00% |
| UDMA | 20.00% |
| DSDM | 20.00% |
| TriEDMA solution | 40.00% |
| Total | 100.00% |

TABLE 22
Powder

| Component | Concentration |
|---|---|
| Quartz 5 μm (RC) | 87.25% |
| OX-50 (RC) | 4.00% |
| R972 | 8.00% |
| NaF | 0.75% |
| Total | 100.0% |

The paste composition L (Quickcure) was prepared by mixing 78.00% powder and 22.00% resin at room temperature. Testing of samples showed an Ortho SBS with Power slot 5s/5s value of 22.22±1.9 MPa, and an Ortho SBS with Normal Light Guide value of 20.68±4.2 MPa.

### Example 16

### Preparation and Testing of Composition M Lacking TPO and CQ

A composition was prepared lacking photoinitiators TPO and CQ, but otherwise similar to Composition J (Example 13). EDMAB was still included as a hydrogen donor.

The composition did not polymerize to any significant extent after 20 seconds of light cure. Hardness values could not be determined. Additionally, there was no significant bleaching of color that would have been associated with polymerization. According to these results, the presence of a photoinitiator other than the color change indicator appears to be required for both polymerization and for a change in color and opacity to be observed.

### Example 17

### Preparation and Testing of Composition N

The following Table provides the components of example composition N.

TABLE 23
Composition N

| Component | Concentration |
|---|---|
| T-4000 [RC] | 43.368% |
| Quartz 5 micron [RC] | 14.110% |
| OX-50 [RC] | 12.450% |
| Fiberglass [RC] | 8.300% |
| UDMA | 6.800% |
| TriEDMA | 6.370% |
| Aerosil R972 | 4.150% |
| BisGMA | 3.400% |
| Sodium fluoride | 0.623% |
| EDMAB | 0.204% |
| UV-3000 | 0.136% |
| CQ | 0.034% |
| TPO | 0.034% |
| H-NU 635 | 0.020% |
| BHT | 0.002% |
| Total | 100.0% |

Color values and opacity were measured for composition N both before and after photo-curing. The following values were observed.

TABLE 24
Test results for composition N

| | L | a | b | Appearance | Opacity |
|---|---|---|---|---|---|
| Uncured | 36.03 | −15.35 | −14.76 | k blue | 93.16% |
| Cured | 62.78 | −2.59 | 8.29 | Vitashade | 76.69% |
| | | | | C2 or A1 | |

The results shown in Table 21 demonstrate that the composition became less opaque after curing (lowered opacity value) and became less colored to finally resemble tooth color after curing. Vitashade C2 has an L value of 68.9, an a value of −0.9, and a b value of 9.7. Vitashade A1 has an L value of 69.3, an a value of −1.0, and a b value of 10.7. The calculated ΔE value for the composition (comparing the uncured and cured composition) is 35.6.

### Example 18

### Evaluation of the Change in Opacity of Compositions after Polymerization

Composition B (Example 5) was polymerized for 160 seconds with 500 mW/cm² intensity light. Samples taken at 10 seconds, 20 seconds, 30 seconds, 40 seconds, 80 seconds, and 160 seconds polymerization were analyzed for their Barcol hardness and opacity values.

TABLE 25
Test results for composition B

| Curing time (sec) | Opacity | Hardness (top) | Hardness (bottom) |
|---|---|---|---|
| 0 | 77.5% | — | — |
| 10 | 61.4% | — | — |
| 20 | 51.4% | 65 | 75 |
| 30 | 38.2% | 68 | 74 |
| 40 | 37.6% | 70 | 75 |
| 80 | 36.4% | 75 | 79 |
| 160 | 36.1% | 78 | 80 |

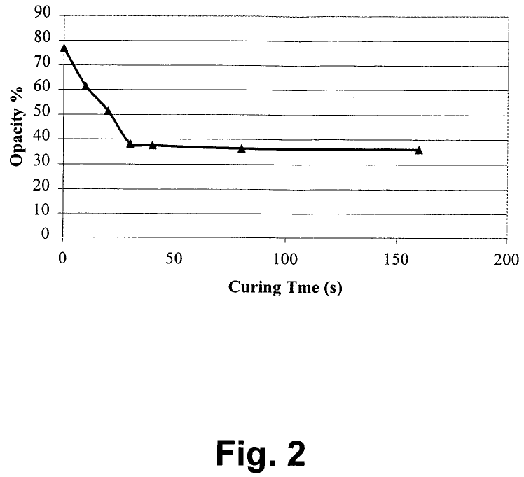

These results show that an approximately 39% percent reduction in opacity (i.e., the composition after polymerization had about 49% of the initial opacity; 38.2/77.5*100%=49%) was obtained within about 30 seconds, and remained fairly constant thereafter. This change in opacity is graphically represented in FIG. 2.

### Example 19

### Use of a Color Change Composition in an Orthodontic Bracket Application

The compositions of Examples 13-15 (Compositions J, K, and L) are suitable for use in orthodontic brackets.

### Example 20

### Use of a Color Change Composition in a Dental Composite Application

The compositions of Examples 1-12 (Compositions A-I) are suitable for use as dental composites.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

1. A colored dental or bone polymerizable composition comprising:
a resin component;
a filler component;
a polymerization initiator; and
a colored polymerization indicator, wherein:
the color intensity of the composition after polymerization is less than the color intensity of the composition before polymerization; and
the opacity of the composition after polymerization is less than the opacity of the composition before polymerization.

2. The composition of claim 1, wherein the color intensity of the composition after polymerization and the color intensity of the composition before polymerization correspond to a ΔE of at least about 30.

3. The composition of claim 1, wherein the opacity of the composition after polymerization is less than about 90% of the opacity of the composition before polymerization.

4. The composition of claim 1, wherein the opacity of the composition after polymerization is less than about 50% of the opacity of the composition before polymerization.

5. The composition of claim 1, wherein the resin component is present at a concentration of about 10 weight percent to about 90 weight percent of the composition.

6. The composition of claim 1, wherein the filler component is present at a concentration of about 10 weight percent to about 90 weight percent of the composition.

7. The composition of claim 1, wherein the resin component comprises a methacrylate monomer, a dimethacrylate monomer, or a multi-methacrylate monomer.

8. The composition of claim 1, wherein the resin component comprises an acrylate monomer, a diacrylate monomer, or a multi-acrylate monomer.

9. The composition of claim 1, wherein the colored polymerization indicator is
6-butoxy-2,4-diiodo-xanthen-3-one,
6-hydroxy-2,4,5,7-tetraiodo-xanthen-3-one, or
6-hydroxy-2,4,5,7-tetraiodo-3-oxo-3H-xanthene-9-carbonitrile.

10. The composition of claim 1, wherein the colored polymerization indicator is present at a concentration of about 0.001 weight percent to about 1 weight percent of the composition.

11. The composition of claim 1, wherein the polymerization initiator is camphorquinone.

12. The composition of claim 1, wherein the composition before polymerization is a color other than bone color or tooth color.

13. The composition of claim 1, wherein the composition before polymerization is red colored, pink colored, orange colored, green colored, blue colored, or purple colored.

14. The composition of claim 1, wherein the composition after polymerization is tooth colored or bone colored.

15. The composition of claim 1, further characterized as being radio-opaque before polymerization, after polymerization, or both before polymerization and after polymerization.

16. The composition of claim 1, further comprising a fixed dye.

17. The composition of claim 1, further comprising an antioxidant.

18. The composition of claim 1, further comprising a fluoride release agent.

19. The composition of claim 1, further comprising a solvent.

20. The composition of claim 1, further comprising water, ethanol, acetone, or mixtures thereof.

21. A dental or bone polymerized composition prepared by polymerizing an unpolymerized composition comprising:
a resin component;
a filler component;
a polymerization initiator; and
a colored polymerization indicator; wherein:
the color intensity of the polymerized composition is less than the color intensity of the unpolymerized composition; and
the opacity of the polymerized composition is less than the opacity of the unpolymerized composition.

22. The polymerized composition of claim 21, wherein the color intensity of the polymerized composition and the color intensity of the unpolymerized composition correspond to a ΔE of at least about 30.

23. The polymerized composition of claim 21, wherein the opacity of the composition after polymerization is less than about 90% of the opacity of the composition before polymerization.

24. The polymerized composition of claim 21, wherein the opacity of the composition after polymerization is less than about 50% of the opacity of the composition before polymerization.

25. The polymerized composition of claim 21, wherein the resin component is present at a concentration of about 10 weight percent to about 90 weight percent of the unpolymerized composition.

26. The polymerized composition of claim 10, wherein the filler component is present at a concentration of about 10 weight percent to about 90 weight percent of the unpolymerized composition.

27. The polymerized composition of claim 21, wherein the resin component comprises a methacrylate monomer, a dimethacrylate monomer, or a multi-methacrylate monomer.

28. The polymerized composition of claim 21, wherein the resin component comprises an acrylate monomer, a diacrylate monomer, or a multi-acrylate monomer.

29. The polymerized composition of claim 21, wherein the colored polymerization indicator is
6-butoxy-2,4-diiodo-xanthen-3-one,
6-hydroxy-2,4,5,7-tetraiodo-xanthen-3-one, or
6-hydroxy-2,4,5,7-tetraiodo-3-oxo-3H-xanthene-9-carbonitrile.

30. The polymerized composition of claim 21, wherein the colored polymerization indicator is present at a concentration of about 0.001 weight percent to about 1 weight percent of the unpolymerized composition.

31. The polymerized composition of claim 21, wherein the polymerization initiator is camphorquinone.

32. The polymerized composition of claim 21, wherein the unpolymerized composition is red colored, pink colored, orange colored, green colored, blue colored, or purple colored.

33. The polymerized composition of claim 21, wherein the polymerized composition is tooth colored or bone colored.

34. The polymerized composition of claim 21, further comprising a radio-opaque component.

35. The polymerized composition of claim 21, further comprising a fixed dye.

36. The polymerized composition of claim 21, further comprising an antioxidant.

37. The polymerized composition of claim 21, further comprising a fluoride release agent.

38. The polymerized composition of claim 21, further comprising a solvent.

39. The polymerized composition of claim 21, further comprising water, ethanol, acetone, or mixtures thereof.

40. A method of treating a dental or bone substrate, the method comprising:
providing the substrate;
providing an unpolymerized composition comprising a resin component, a filler component, a polymerization initator, and a colored polymerization indicator;
applying the unpolymerized composition to the substrate; and
polymerizing the unpolymerized composition to prepare a polymerized composition, wherein:
the color intensity of the polymerized composition is less than the color intensity of the unpolymerized composition; and
the opacity of the polymerized composition is less than the opacity of the unpolymerized composition.

41. The method of claim 40, wherein the substrate is a tooth, a dental implant, an artificial tooth, or a bone.

42. The method of claim 40, wherein the color intensity of the polymerized composition and the color intensity of the unpolymerized composition correspond to a ΔE of at least about 30.

43. The method of claim 40, wherein the opacity of the composition after polymerization is less than about 90% of the opacity of the composition before polymerization.

44. The method of claim 40, wherein the opacity of the composition after polymerization is less than about 50% of the opacity of the composition before polymerization.

45. The method of claim 40, wherein the resin component is present at a concentration of about 10 weight percent to about 90 weight percent of the unpolymerized composition.

46. The method of claim 40, wherein the filler component is present at a concentration of about 10 weight percent to about 90 weight percent of the unpolymerized composition.

47. The method of claim 40, wherein the resin component comprises a methacrylate monomer, a dimethacrylate monomer, or a multi-methacrylate monomer.

48. The method of claim 40, wherein the resin component comprises an acrylate monomer, a diacrylate monomer, or a multi-acrylate monomer.

49. The method of claim 40, wherein the colored polymerization indicator is
6-butoxy-2,4-diiodo-xanthen-3-one,
6-hydroxy-2,4,5,7-tetraiodo-xanthen-3-one, or
6-hydroxy-2,4,5,7-tetraiodo-3-oxo-3H-xanthene-9-carbonitrile.

50. The method of claim 40, wherein the colored polymerization indicator is present at a concentration of about 0.001 weight percent to about 1 weight percent of the unpolymerized composition.

51. The method of claim 40, wherein the polymerization initiator is camphorquinone.

52. The method of claim 40, wherein the unpolymerized composition is red colored, pink colored, orange colored, green colored, blue colored, or purple colored.

53. The method of claim 40, wherein the polymerized composition is tooth colored or bone colored.

54. The method of claim 40, wherein the unpolymerized composition is radio-opaque, the polymerized composition is radio-opaque, or both the unpolymerized composition and the polymerized composition are radio-opaque.

55. The method of claim 40, wherein the unpolymerized composition further comprises a fixed dye.

56. The method of claim 40, wherein the unpolymerized composition further comprises an antioxidant.

57. The method of claim 40, wherein the unpolymerized composition further comprises a fluoride release agent.

58. The method of claim 40, wherein the unpolymerized composition further comprises a solvent.

59. The method of claim 40, wherein the unpolymerized composition further comprises water, ethanol, acetone, or mixtures thereof.
